(19) United States
(12) Patent Application Publication
(10) Pub. No.: US 2020/0390481 A1
(43) Pub. Date: Dec. 17, 2020

(54) Femoral Fracture Fixation System

(71) Applicant: Stryker European Operations Limited, Carrigtwohill (IE)

(72) Inventor: Benjamin Wünsch, Dusseldorf (DE)

(21) Appl. No.: 16/826,682

(22) Filed: Mar. 23, 2020

Related U.S. Application Data
(60) Provisional application No. 62/861,017, filed on Jun. 13, 2019.

(51) Int. Cl. A61B 17/74

(52) U.S. Cl. CPC A61B 17/744; A61B 17/748

(57) ABSTRACT

An implant system includes an intramedullary nail that has a proximal and distal portions. The proximal portion defines a central bore extending along a central axis of the proximal portion. The central bore defines a first camming surface. The intramedullary nail defines a transverse bore that extends transverse to the central axis of the proximal portion and intersects the central bore. A bone fastener is disposed within the transverse bore and has a groove formed on an outer surface. Also, a coupling insert is disposed within the central bore and is moveable along the central axis. The coupling insert has a first portion engaged to the central bore and a second portion that has a first moveable member extending from the first portion. Upon moving the coupling insert along the central axis, the first camming surface engages the first movable member to deflect the first moveable member into alignment with the groove of the bone fastener.

### CROSS-REFERENCE TO RELATED APPLICATIONS

This application claims the benefit of the filing date of U.S. Provisional Patent Application No. 62/861,017, filed Jun. 13, 2019, the disclosure of which is hereby incorporated herein by reference.

### BACKGROUND OF THE INVENTION

The present disclosure generally relates to an implant system for use in orthopedic surgery. Specifically, the disclosure relates to an intramedullary nail for internal fixation of bone, such as a femur.

Femur fractures commonly occur in the femoral neck and the trochanteric regions. Typically, trochanteric and sub-trochanteric femur fractures are treated with an intramedullary nail having a transverse bore to receive a bone fastener, such as a femoral neck screw usually provided in the form of a lag screw.

U.S. Pat. No. 5,176,681, which is incorporated by reference herein in its entirety, discloses an intramedullary nail that is adapted to be inserted into an intramedullary canal of a femur and a lag screw that is adapted to be passed through a transverse bore of the intramedullary nail, through the neck of the femur, and into the femoral head. The lag screw is designed to transfer the load of the femoral head into the nail shaft by bridging the fracture line to allow fast and secure fracture healing. Further, the lag screw is allowed to slide in the intramedullary nail so as to accommodate settling of the fracture. A set screw that is inserted into a longitudinal bore of the intramedullary nail engages the lag screw to prevent rotation and uncontrolled medial deviation of the lag screw while allowing for the sliding movement thereof. The intramedullary nail includes a central cannulation along its longitudinal axis for receiving a surgical wire (guidewire), such as a Kirschner-wire. The surgical wire is inserted into the marrow cavity of the femur prior to the insertion of the intramedullary nail.

However, conventional intramedullary nails have several drawbacks, one of which being that conventional set screws occlude the central cannulation of the intramedullary nail. Thus, conventional set screws cannot be preassembled with the intramedullary nail and thus have to be inserted into the intramedullary nail intraoperatively after removal of a guidewire. In this case, the insertion of the relatively small set screw into the shaft of the intramedullary nail is cumbersome. Soft tissue overlapping the opening at the proximal end of the nail may hinder the insertion of the set screw and the mutual engagement of the threads. Thus, the set screw may get stuck within the intramedullary nail and the operation time may be increased due to additional operation steps. There is also the additional concern that misthreading of the set screw with the intramedullary nail can damage the set screw or the intramedullary nail. Moreover, another drawback is that conventional set screws are susceptible to backing out which can negatively affect the performance of the lag screw. Therefore, further improvements are desirable.

### BRIEF SUMMARY OF THE INVENTION

In one aspect of the present disclosure, an implant system for fixation of bone, includes a bone fastener that has a groove formed on an outer surface thereof. An intramedullary nail includes a proximal portion and a distal portion. The proximal portion defines a central bore extending along a central axis of the proximal portion. The intramedullary nail defines a transverse bore that extends transverse to the central axis of the proximal portion and intersects the central bore. The transverse bore is configured to receive the bone fastener therein. A coupling insert is configured to be received within the central bore and has first and second portions spaced along a longitudinal axis of the coupling insert. The coupling insert defines a through-opening extending along the longitudinal axis through the first and second portions. The second portion includes a plurality of moveable members extending from the first portion and terminating at respective terminal ends. The moveable members are moveable relative to each other such that the terminal ends of the moveable members are separated from one another in a first configuration and relatively closer together in a second configuration, such that in the second configuration, the terminal ends of the moveable members at least partially occlude the through-opening and are at least partially receivable within the groove of the bone fastener when the bone fastener is disposed within the transverse bore.

In another aspect of the present disclosure, an implant system for fixation of bone includes an intramedullary nail that has a proximal portion and a distal portion. The proximal portion includes a central bore extending along a central axis of the proximal portion. The central bore defines a first camming surface. The intramedullary nail defines a transverse bore that extends transverse to the central axis of the proximal portion and intersects the central bore. A bone fastener is disposed within the transverse bore and has a groove formed on an outer surface thereof. A coupling insert is disposed within the central bore and is moveable along the central axis. The coupling insert has a first portion engaged to the central bore and a second portion has a first moveable member that extends from the first portion. Upon moving the coupling insert along the central axis, the first camming surface engages the first movable member to deflect the first moveable member into alignment with the groove of the bone fastener.

In a further aspect of the present disclosure, a method of securing a bone fracture includes: inserting an intramedullary nail and a coupling insert disposed within a proximal portion of the intramedullary nail distally into a femur along a guidewire; removing the guidewire proximally from the intramedullary nail and the coupling insert, such that the guidewire passes through a central cannulation of the nail and through a through-opening of the coupling insert; driving a bone fastener through a transverse bore in the intramedullary nail; and moving the coupling insert distally toward the bone fastener causing a plurality of leg members of the coupling insert to be displaced from a radially expanded position to a radially contracted position and positioning terminal ends of the leg members at least partially within a groove in the bone fastener so as to prohibit rotation of the bone fastener.

In an additional aspect of the present disclosure, a coupling insert for use with an implant system for fixation of bone includes a first portion and a second portion spaced from the first portion along a longitudinal axis. The second portion includes a plurality of moveable members extending from the first portion and terminating at respective terminal ends. The moveable members being moveable relative to each other such that the terminal ends of the moveable members are separated from one another in a first configuration and relatively closer together in a second configuration. A through-opening extends along the longitudinal axis through the first and second portions. In the second configuration, the terminal ends of the moveable members at least partially occlude the through-opening.

In a yet further aspect of the present disclosure, a method of securing a bone fracture includes: moving a coupling insert within a proximal portion of an intramedullary nail distally toward a bone fastener disposed within a transverse bore in the intramedullary nail, causing a plurality of leg members of the coupling insert to be displaced from a radially expanded position to a radially contracted position and positioning terminal ends of the leg members at least partially within a groove in the bone fastener so as to prohibit rotation of the bone fastener.

### BRIEF DESCRIPTION OF THE DRAWINGS

The features, aspects, and advantages of the present invention will become better understood with regard to the following description, appended claims, and accompanying drawings in which:

FIG. 1 is an implant system according to one embodiment of the present disclosure within a femur.

FIG. 2 is a detailed view of a proximal portion of the implant system of FIG. 1.

FIG. 3A is a perspective view of a coupling insert of the implant system of FIG. 1.

FIG. 3B is an elevational view of the coupling insert of FIG. 3A.

FIG. 3C is a cross-sectional view of the coupling insert of FIG. 3A taken along a midline thereof.

FIG. 3D is a bottom view of the coupling insert of FIG. 3A.

FIGS. 4A-4E are cross-sectional views of the implant system of FIG. 1 taken along a plane bisecting an intramedullary nail of the implant system in an anteroposterior direction and demonstrating a method of use.

FIG. 4F is cross-sectional view of the implant system of FIG. 1 taken along a plane bisecting the implant system in a medial-lateral direction.

### DETAILED DESCRIPTION

When referring to specific directions in the following discussion of certain implantable devices, it should be understood that such directions are described with regard to the implantable device's orientation and position during exemplary application to the human body. Thus, as used herein, the term “proximal” means close to the heart and the term “distal” means more distant from the heart. The term “inferior” means toward the feet and the term “superior” means toward the head. The term “anterior” means toward the front of the body or the face, and the term “posterior” means toward the back of the body. The term “medial” means toward the midline of the body, and the term “lateral” means away from the midline of the body. Also, as used herein, the terms “about,” “generally” and “substantially” are intended to mean that slight deviations from absolute are included within the scope of the term so modified.

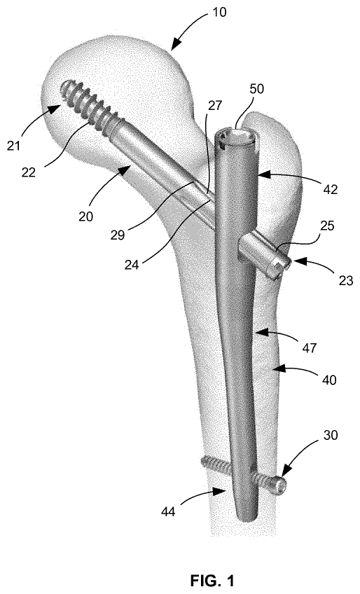

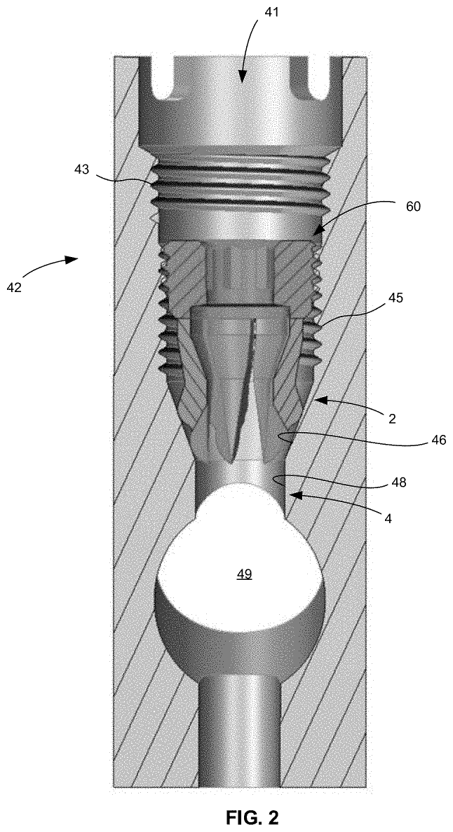

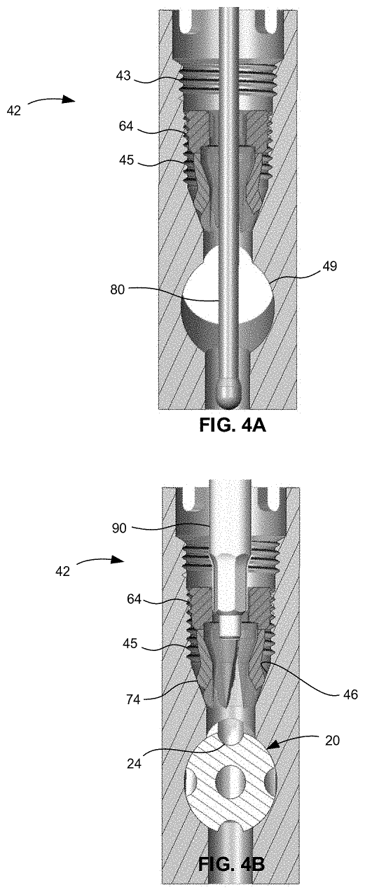

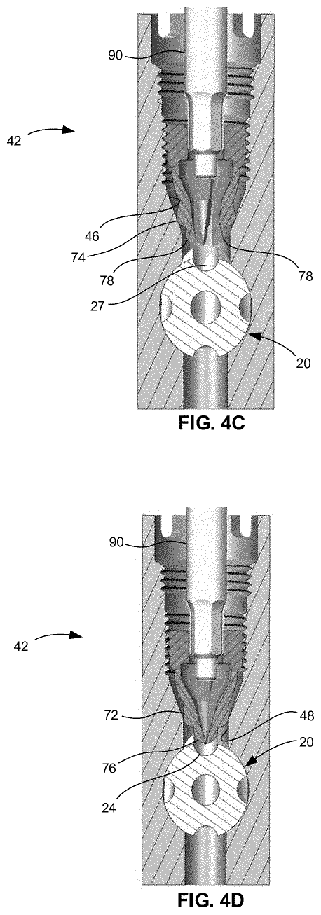

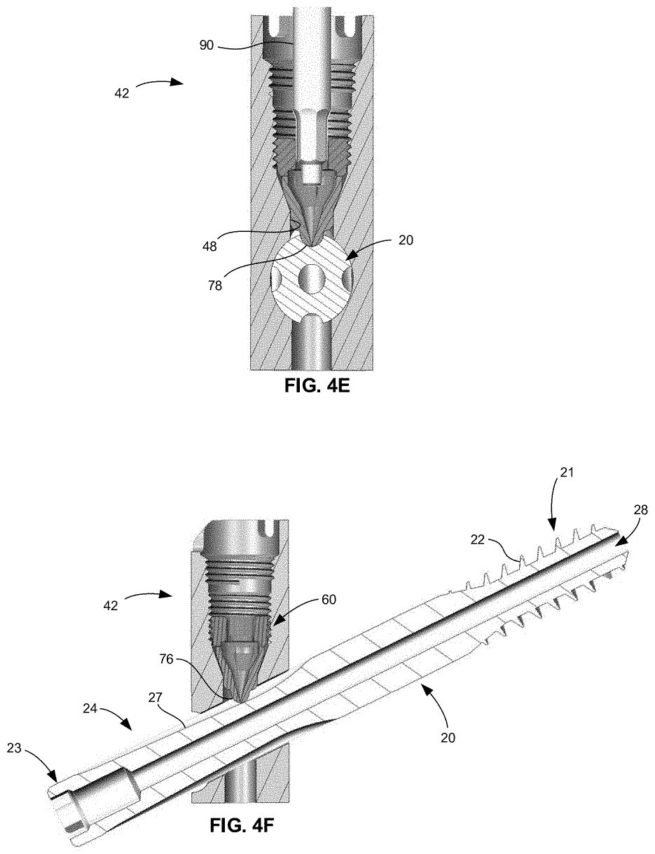

FIGS. 1 and 2 depict an implant system for fixation of bone, such as a femur 10. The implant system generally includes a lag screw 20, one or more bone screws 30, an intramedullary nail 40, an end cap 50, and a coupling insert 60. Lag screw or bone fastener 20 is a femoral neck screw that includes a leading end portion 21 that includes a thread 22, for example a coarse thread, and a trailing end portion 23. Trailing end portion 23 includes a plurality of longitudinally extending grooves 24 formed in an outer surface of lag screw 20 and arranged about a longitudinal axis thereof at predefined intervals. In the embodiment depicted, four grooves 24 are arranged at intervals of 90 degrees about the longitudinal axis of lag screw 20. However, in other embodiments more or less grooves 24 may be provided. Each groove 24 is defined by a ramped surface 27 that gradually declines from a shallow end 25 to a deeper end 29. In the depicted lag screw 20, the deeper end 29 is closer than the shallow end 25 to the leading end 21 of lag screw 20. Thus, grooves 24 gradually become deeper from the trailing end 23 toward the leading end 21 of lag screw 20. Further, lag screw 20 includes a cannulation 28 extending along the longitudinal axis thereof, as best shown in FIG. 4F. Trailing end 23 of lag screw is configured to engage a tool such as a screw driver or wrench (e.g., in the form of an entrained driving feature).

Intramedullary nail 40 is a rod-shaped body that includes a proximal portion 42, a distal portion 44, and an intermediate bent portion 47. In other words, the intermediate bent portion 47 connects proximal portion 42 and distal portion 44. Distal portion 44 includes one or more screw openings for bone screw 30. Intramedullary nail 40 also includes a central cannulation or central longitudinal bore 41 that extends along and through the entire length of nail 40, and a transverse bore 49 that extends entirely through nail 42 in a direction transverse to a longitudinal axis of central bore 41. In this regard, transverse bore 49 intersects central bore 41 and is preferably angled relative to the central axis of central bore 41 at an oblique angle.

Proximal portion 42 of nail 40, as best shown in FIG. 2, is configured to receive end cap 50 and coupling insert 60. In this regard, proximal portion 42 includes a cap engagement structure and a coupling insert guiding structure. Such structures define the proximal end of central bore. The cap engagement structure includes a first threaded section 43 which is configured to threadedly engage end cap 50. The guiding structure includes a second threaded section 45, a conical section 2, and a cylindrical section 4. Threaded section 45 is configured to threadedly engage coupling insert 60, as described below. Conical section 2 is defined by a conical surface or camming surface 46. The cylindrical section 4 is defined by a cylindrical surface 48. In the embodiment depicted, conical surface 46 is disposed between cylindrical surface 48 and second threaded section 45. In addition, cylindrical surface 48 intersects transverse bore 49 such that the guiding structure is in direct communication with transverse bore 49 and lag screw 20 when disposed therein. However, in other embodiments, additional sections of differing or similar surface geometries may be provided. For example, a non-threaded cylindrical surface (not shown) can be located between second threaded section 45 and conical surface 46, for example.

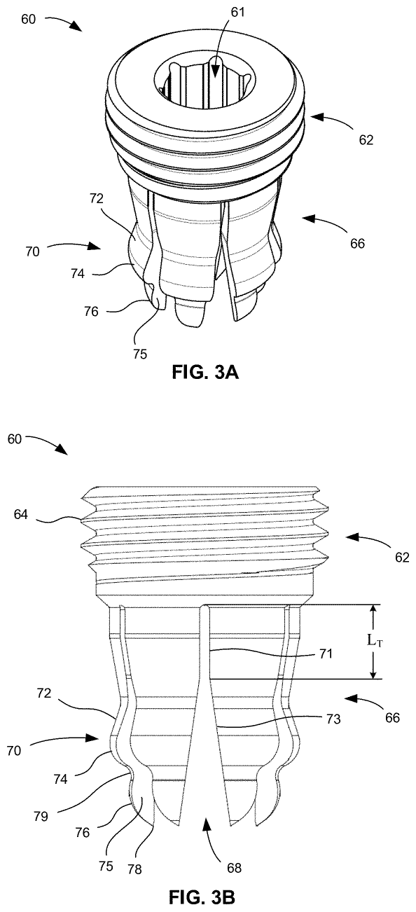

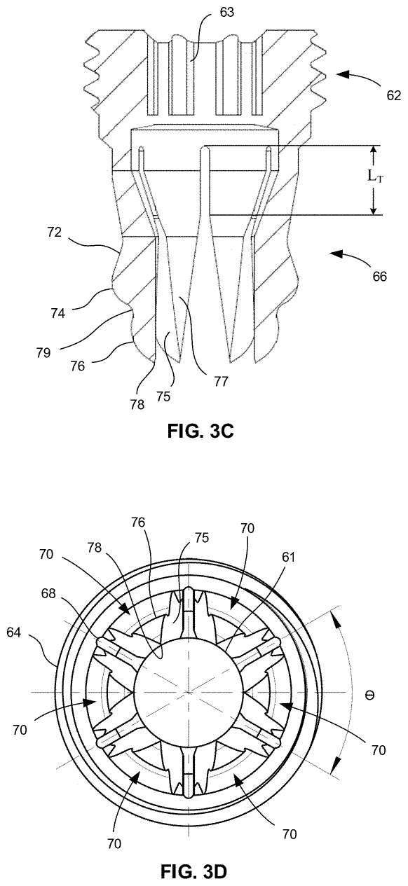

FIGS. 3A-3D depict coupling insert 60. Coupling insert 60 generally includes a first portion or head portion 62 and a second portion or screw engagement portion 66. In addition, coupling insert 60 defines a through-opening 61 extending entirely therethrough such that insert 60 is cannulated to receive a guidewire therethrough.

Head portion 62 is a ring-shaped or annular structure that includes an inner tool engagement surface 63 which is configured to engage a tool, such as a screw driver, hex driver, or the like. In addition, head portion 62 has an outer thread 64 extending about a longitudinal axis of head portion 62 and along its length. Thread 64 is configured to engage second threaded portion 45 of intramedullary nail 40.

Screw engagement portion 66 includes a plurality of moveable members or legs 70. Legs 70 are cantilevered to head portion 62 such that they each extend from a fixed end at head portion 62 to a terminal end 78. Legs 70 are integrated into head portion 62 such that head portion 62 and legs 66 form a monolithic or unitary structure.

Each leg 70 is separated from an adjacent leg 70 via an inter-leg slot 68 and from an opposed leg 70 by through-opening 61. Each slot 68 is defined by a pair of legs 70 such that slot 68 has a constant width portion 71 and a tapered portion 73. In another embodiment, slot 68 may define a keyhole shape such that the constant width portion 71 is instead a circular portion. Constant width portion 71 is located closer to head portion 62 than tapered portion 73. Tapered portion 73 tapers outwardly in a direction away from head portion 62 and terminates at terminal ends 78 of legs 70. In the embodiment depicted, there are six legs 70 which are circumferentially arranged about the longitudinal axis of coupling insert 60 at evenly spaced intervals such that legs 70 and slots 68 between them are circumferentially offset from each other at an angle θ. In the embodiment depicted, angle θ is 60 degrees. However, in other embodiments where there are more or less than six legs 70, angle θ may differ from that shown. For example, in embodiments where coupling insert 60 includes four legs 70, angle θ may be 90 degrees. In another example, where coupling insert 60 includes eight legs 70, angle θ may be 45 degrees.

Each leg 70 includes an outer surface, an inner surface 77, and side surfaces 75 extending therebetween. The outer surfaces of each leg 70 include cylindrically-curved surface 72, a first convex or bulbous surface 74, a second convex or bulbous surface 76, and a concave or indented surface 79. Cylindrically-curved surface 72, as described in more detail below, defines a cylinder with the remaining legs 70 when the legs 70 are brought together. Thus, cylindrically-curved surface 72 is curved in only one plane and about an axis. In contrast, bulbous surfaces 74 and 76 are curved in more than one plane. First bulbous surface 74 is located between cylindrically-curved surface 72 and indented surface 79. In addition, indented surface 79 is positioned between first bulbous surface 74 and second bulbous surface 76. Second bulbous surface 76 partially defines the terminal end 78 of leg 70. First and second bulbous surfaces 74, 76 each have a radius of curvature. For example, the radius of curvature of first bulbous surface 74 may be 1 mm, while the radius of curvature of the second bulbous surface 76 may be 1.5 mm. In this regard, the radius of curvature of first bulbous surface 74 may be smaller than that of second bulbous surface 76. Moreover, first bulbous surface 74 protrudes further radially outwardly than second bulbous surface 76.

Side surfaces 75 of each leg taper toward each other in a direction toward the longitudinal axis of coupling insert 60 and toward inner surface 77. In addition, the outer surface and inner surface 77 of each leg 70 each have a width that extends from one side surface 75 to the other. Such width tapers in a proximal-distal direction, which when combined with the curvatures of second bulbous surface 76, results in legs 70 narrowing to a point at terminal end 78, as best shown in FIGS. 3B and 3C. However, the proximal-distal taper of the width of the outer surface and inner surface 77 begins at a transition region located at a length L_{T} from the fixed end of leg 70. Length L_{T} may be about a quarter to three-quarters of the total length of the leg 70. This transition region also coincides with the transition of each slot 68 from the constant width portion 71 to the tapered portion 73, as best shown in FIG. 3B. Thus, the outer surface and inner surface 77 have a constant width proximal of the transition region and a tapering width distal of the transition region.

When insert 60 is at rest, legs 70 are biased toward a first position or expanded configuration is shown in FIGS. 3A and 3B. However, legs 70 are moveable to a second position or contracted configuration in which terminal ends 78 of legs 70 come together and sides surfaces contact each other (see FIG. 4E). This coming together is facilitated by the shape of the longitudinal slots 68 between legs 70. When legs 70 are in their second position, they occlude through-opening 61 at the distal end of insert 60. Thus, legs 70 facilitate the passage of a guidewire through through-opening 61 when in the first position, but then obstruct, at least partially, through-opening 61 when in the second position and when the guidewire is removed. However, legs 70, when in the second position, can be positioned with a groove 24 of lag screw 20, as described in more detail below. In this regard, first bulbous surface 74 is a camming surface that is operable with camming surface 46 of nail 40 to facilitate movement of legs 70 toward the second position as insert 60 is moved distally within proximal portion 42 of nail 40. In addition, second bulbous surfaces 74 of legs 70 come together to form an approximate spherically-shaped tip or rounded nub that is conformingly received within the concave groove 24 of lag screw 20 to prevent rotation thereof about its longitudinal axis while positioned within the transverse bore 49. The spherically-shaped tip formed by the coming together of legs 70 is described as “approximate” because an actual spherical shape is unlikely due at least to the deformation of arms 70. Although, it should be understood that, in other embodiments, other shapes may be approximated by legs 70, such as cylindrical or rectangular, for example. Generally, though, the approximated shape preferably at least substantially matches the contours of the elongate groove 24 of lag screw. Moreover, cylindrically-curved surfaces 72 of each leg 70 together define an approximate cylindrical shape or form when insert 60 is in the second position which is receivable within cylindrical section 4 of nail 40. Again, surfaces 72 are described as coming together to form an “approximate” cylindrical shape as deformation of arms 70 make a perfect cylindrical shape unlikely. Although, again, other shapes are possible, such as a rectangle, for example. The bias of legs 70 causes legs 70 to bear on cylindrical surface 48 to create frictional resistance to back-out of coupling insert 60, as best shown in FIG. 4E and as described below.

FIGS. 4A-4F depict a method of using the implant system described above. Implant system may be used to treat trochanteric and sub-trochanteric femur fractures. In this regard, an operator gains access to a patient's proximal femur and creates an entry point by opening the femoral cortex at the greater trochanter or piriformis fossa. A guidewire 80 may then be inserted into the intramedullary canal of the femur 10. A reamer may then be used over the guidewire to prepare the intramedullary canal for receipt of nail 40.

Once the intramedullary canal is prepared and with guidewire 80 disposed within and extending from the proximal femur, nail 40 is advanced over guidewire 80 and into the intramedullary canal. Nail 40 is preferably provided with coupling insert 60 pre-assembled with nail 40. In this regard, coupling insert 60 is shipped to the operating theater with the coupling insert 60 already inserted into central bore 41. Alternatively, coupling insert 60 is inserted into bore 41 of nail 40 in the operating theater prior to insertion into femur 10. This may be done by using a driver to thread head portion 62 of coupling insert 60 to second threaded section 45 of nail 40. In this regard, first threaded section 43 is generally of a larger cross-sectional dimension than that of second threaded section 45 such that coupling insert 60 is passed through first threaded section 43 without engagement of threads 64 therewith. However, in some embodiments, first and second threaded sections 43, 45 may have the same cross-sectional dimension or they may blend together to form a single, longer threaded section. Regardless of where coupling insert 60 is inserted into bore 41, it is preferably done prior to insertion of nail 40 into the patient's femur 10. This helps ensure threads 64 of coupling insert 60 are properly engaged with second threaded section 45 of nail 40. It also helps simplify the procedure as the operator does not have to insert coupling insert 60 into nail 40 while nail 40 is in the patient's bone 10. Thus, once nail 40 is inserted into femur 10, guidewire 80 passes through central bore 41 of nail 40 and also through through-opening 61 of coupling insert 60, as best shown in FIG. 4A. This is possible at least because legs 70 are in an expanded, unloaded state when in their first position such that through-opening 61 is clear of obstruction and, therefore, capable of receiving guidewire 80. Thus, pre-loading of nail 40 with coupling insert 60 is done such that coupling insert 60 is not inserted too deep within bore 41 in order to ensure through-opening 61 remains clear for guidewire 80.

In order to help ensure that coupling insert 60 is not inserted too deeply when pre-loading nail 40, head portion 62 may be configured such that a limited number of turns of coupling insert 60 relative to second threaded section 45 can be achieved to couple insert 60 with nail 40 without moving legs 70 or without moving legs 70 too much that guidewire 80 cannot be advanced therethrough. For example, threads 64 may be configured such that 1 to 3 complete turns of coupling insert 60 would be effective to pre-load insert 60 without concerns that insert would move out of engagement with threaded section 45 during transport and implantation and that through-opening 61 would be obstructed. In another embodiment, a recess (not shown) may be positioned between first and second threaded sections 43, 45, and first and second threaded sections 43, 45 may both be engageable with threads 64 of head portion 62. In this regard, head portion 62 can be threaded through first threaded section 43 and into the recess between first and second threaded sections 43, 45 so as to secure insert 60 while nail 40 is transported and implanted. In such position, legs would be in their expanded configuration. Thereafter, insert 60 can be advanced from the recess and into engagement with second threaded section 45.

Thereafter, guidewire 80 is removed by advancing guidewire 80 through nail 40 and coupling insert 60 and out of femur 10. At this point, lag screw 20 may be passed through transverse bore 49, across the fracture, and into the femoral neck and head. Lag screw 20 is rotated so that one of its grooves 24 is aligned with central bore 41 of nail and faces superiorly toward coupling insert 60, as shown in FIG. 4B.

With the lag screw 20 properly inserted, a driver 90 is inserted into central bore 41 and into through-opening 61 of coupling insert 60 so that an operative end of driver 90 engages tool surface 63 within head portion 62, as also shown in FIG. 4B. Thereafter, driver 90 is rotated so as to advance coupling insert 60 along second threaded section 45 and along the central axis of proximal portion 42. As coupling insert 60 is advanced through central bore 41 toward lag screw 20, first and second camming surfaces 46, 74 come into engagement which results in an inwardly applied force on each leg 70 such that terminal ends 78 of each leg 70 are displaced toward each other and toward the central axis of proximal portion 42, as shown in FIGS. 4B-D.

Continued advancement of coupling insert 60 eventually results in the legs 70 coming together and contacting each other such that terminal ends 78 of each leg 70 are aligned with groove 24 of nail 20 and such that second bulbous surface 76 of each leg come together to form the rounded nub, which is configured to match the curvature of groove 24, as shown in FIG. 4D. In addition, terminal ends 78 of legs 70 at the distal end of insert 60 obstruct through-opening 61 such that guidewire 80 could not be positioned therein. Thus, through-opening 61 is closed at the distal end of insert 60 when legs 70 are in their second position. However, through-opening 61 remains open at the proximal end of insert 60 so that driver 90 can be moved in and out of through-opening 61 an into engagement with tool surface 63. It should be noted that, while the depicted embodiment completely obstructs or occludes through-opening 61 at the distal end, legs 70 may not come completely together in a way that through-opening 61 is completely obstructed but is obstructed enough that guidewire 80 would be incapable of passing therethrough. However, it is preferable that terminal ends 78 do come into contact with each other which strengthens the distal end of coupling insert 60 so that terminal ends 78 are less susceptible to damage than when they do not contact each other.

At this point, first bulbous surface 74 is advanced beyond conical camming surface 46. It is noted that the narrowest cross-sectional dimension of conical section 2 is such that it allows for legs 70 to be brought together into contact with each other but still allow advancement into cylindrical section 4. In this regard, the cross-sectional dimension of the narrowest part of conical section 2 and also of the cylindrical section 4 is slightly larger than that of coupling insert 60 as measured at the cylindrical-curved surfaces 72 of legs 70 when legs 70 are in their contracted position.

When first bulbous surfaces 74 of legs 70 advance into cylindrical section 4 and legs 70 are brought together, cylindrically-curved surfaces 72 of legs 70 form an approximate cylindrical form that fits within cylindrical section 4. However, due to the bias of legs 70, cylindrically-curved surfaces 72 bear against cylindrical surface 48 creating frictional resistance to back-out of coupling insert 60, as best shown in FIGS. 4D and 4E. However, coupling insert 60 may still be advanced until terminal ends 78 of legs 70 contact ramped surface 27 of groove 24, as shown in FIGS. 4E and 4F. When legs 70 firmly engage ramped surface 27, rotation and linear translation of lag screw 20 is prohibited. However, coupling insert 60 may be backed out slightly so that legs 70 are positioned within groove 24 to prevent rotational movement of lag screw 20 but are not in firm contact with ramped surface 27 so that linear translation is possible. Due to the incline of ramp 27, translation may only occur laterally in one direction so as to facilitate the fusing of bone while preventing the bone fragments from separating.

End cap 50 may then be inserted into central bore 41 and threaded to first threaded section 43. Also, bone screw 30 may be passed through distal portion 44 of nail 40 for further fixation.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. An implant system for fixation of bone, comprising:
a bone fastener having a groove formed on an outer surface thereof;
an intramedullary nail having a proximal portion and a distal portion, the proximal portion defining a central bore extending along a central axis of the proximal portion, the intramedullary nail defining a transverse bore extending transverse to the central axis of the proximal portion and intersecting the central bore, the transverse bore being configured to receive the bone fastener therein; and
a coupling insert configured to be received within the central bore and having first and second portions spaced along a longitudinal axis of the coupling insert, the coupling insert defining a through-opening extending along the longitudinal axis through the first and second portions, the second portion comprising a plurality of moveable members extending from the first portion and terminating at respective terminal ends, the moveable members being moveable relative to each other such that the terminal ends of the moveable members are separated from one another in a first configuration and relatively closer together in a second configuration, such that in the second configuration, the terminal ends of the moveable members at least partially occlude the through-opening and are at least partially receivable within the groove of the bone fastener when the bone fastener is disposed within the transverse bore.

2. The system of claim 1, wherein the central bore of the nail includes a threaded section, and the first portion of the coupling insert includes a thread configured to threadedly engage the threaded section.

3. The system of claim 2, wherein the moveable members are cantilevered to the first portion such that the first portion and the moveable members form a monolithic structure.

4. The system of claim 1, wherein the central bore of the nail defines a first camming surface configured such that when the coupling insert is driven toward the transverse bore, the movable members contact the first camming surface to move the moveable members radially inwardly toward the central axis of the proximal portion.

5. The system of claim 4, wherein the first camming surface is a conical surface.

6. The system of claim 5, wherein each of the movable members includes a bulbous surface extending radially outwardly and configured to contact the conical surface.

7. The system of claim 1, wherein the moveable members are circumferentially arrayed about the longitudinal axis of the coupling insert such that each moveable member is circumferentially offset from an adjacent moveable member at an angle.

8. The system of claim 7, wherein the angle is 60 degrees.

9. The system of claim 1, wherein, in the first configuration, a pair of adjacent moveable members defines a slot therebetween, the slot having a constant width portion and a tapered portion.

10. The system of claim 9, wherein the constant width portion of the slot is positioned closer to the first portion of the coupling insert, and the tapered portion tapers outwardly from the constant width portion to the terminal ends of the pair of moveable members.

11. The system of claim 1, wherein the central bore of the nail includes a threaded section, a conical section, and a cylindrical section, the conical section being disposed between the threaded section and the cylindrical section.

12. The system of claim 11, wherein each of the moveable members includes a cylindrically-curved surface, a first bulbous surface, and a second bulbous surface, the first bulbous surface being positioned between the curved surface and second bulbous surface.

13. The system of claim 12, wherein, in the second configuration, the curved surfaces of the moveable members together define a cylindrical shape that is configured to be received within the cylindrical section of the central bore of the nail.

14. The system of claim 1, wherein, in the second configuration, the moveable members obstruct the through-opening at a distal end of the coupling insert while the through-opening remains unobstructed at a proximal end of the coupling insert so as to be configured to receive a tool.

15. The system of claim 1, wherein the moveable members are biased outwardly from the longitudinal axis in the first configuration such that when the moveable members are in the second configuration, the moveable members bear on a cylindrical surface of the central bore of the nail when disposed within the central bore.

16. An implant system for fixation of bone, comprising:
an intramedullary nail having a proximal portion and a distal portion, the proximal portion defining a central bore extending along a central axis of the proximal portion, the central bore defining a first camming surface, the intramedullary nail defining a transverse bore extending transverse to the central axis of the proximal portion and intersecting the central bore;
a bone fastener disposed within the transverse bore and having a groove formed on an outer surface thereof; and
a coupling insert disposed within the central bore and moveable along the central axis, the coupling insert having a first portion engaged to the central bore and a second portion having a first moveable member extending from the first portion,
wherein, upon moving the coupling insert along the central axis, the first camming surface engages the first movable member to deflect the first moveable member into alignment with the groove of the bone fastener.

17. The system of claim 16, further comprising a second moveable member positioned adjacent the first moveable member.

18. The system of claim 17, wherein the first and second moveable members define a first position in which the first and second members to do not contact each other and a second position in which the first and second members are in contact with each other.

19. The system of claim 17, wherein the first and second moveable members define a slot therebetween, the slot having a constant width portion and a tapered portion.

20. The system of claim 19, wherein the constant width portion of the slot is positioned closer to the first portion of the coupling insert, and the tapered portion tapers outwardly from the constant width portion to terminal ends of the first and second moveable members.
